(19) United States
(12) Patent Application Publication
(10) Pub. No.: US 2007/0116769 A1
(43) Pub. Date: May 24, 2007

(54) Continuous encapsulation process

(75) Inventor: Poul Bach, Birkeroed (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 11/385,451

(22) Filed: Mar. 21, 2006

Related U.S. Application Data
(63) Continuation-in-part of application No. 11/265,061, filed on Nov. 1, 2005.
(60) Provisional application No. 60/630,314, filed on Nov. 23, 2004.

(30) Foreign Application Priority Data
Nov. 1, 2004 (DK) 2004 01674

(51) Int. Cl. C12N 11/04; A61K 9/14

(52) U.S. Cl. 424/489; 435/182; 427/2.14

(57) ABSTRACT

The present invention relates to a process for coating of particles comprising the steps of: feeding particles to a coating unit wherein said particles are coated; directly transferring coated particles from the coating unit to a separator unit; separating finished coated particles from unfinished coated particles; removing the finished coated particles and transferring the unfinished coated particles directly from the separator unit to a holding unit, and directly transferring unfinished coated particles from the holding unit to the coating unit for re-coating.

### CROSS-REFERENCE TO RELATED APPLICATION

This application is a continuation-in-part of U.S. application Ser. No. 11/265,061 filed Nov. 1, 2005, which claims priority or the benefit under 35 U.S.C. 119 of Danish application no. PA 2004 01674 filed Nov. 1, 2004 and U.S. provisional application No. 60/630,314 filed Nov. 23, 2004, the contents of which are fully incorporated herein by reference.

### FIELD OF THE INVENTION

The present invention relates to a high capacity process for coating of particles, with improved ability for controlling product quality, comprising a coating step wherein particles are coated a selection step wherein it is determined whether or not the particles need to be recoated and a holding step wherein the particles can be stored until there is free capacity for the particles to be transferred back to the coating unit. The present invention further relates to coated particles produced by said process. The process consists of recycling particles between at least three units connected in a loop.

### BACKGROUND OF THE INVENTION

It is known in the art to use fluid bed coating apparatuses for coating of particles comprising active compounds such as enzymes, e.g., WO 99/32595.

Conventional fluid bed coaters known in the art are either of the batch type or of the continuous type. The batch fluid bed coater is characterised by processing the same initial load of particles, i.e., no new particles are added or removed during the processing time of the batch. In a continuous fluid bed coater new solid particles are fed into the coater at a constant rate and the corresponding number, of coated particles are continuously removed from the fluid bed coater if no agglomeration takes place.

The batch fluid bed has several advantages over a continuous fluid bed when thin uniform layers have to be applied as all particles have exactly the same residence time (equal to the processing time—also called the batch time). The continuous operating fluid bed coater has a certain residence time distribution, which results in a corresponding distribution of coating thicknesses. In most cases it is an unwanted property leading to inferior product quality. The continuous fluid bed coater has, however, the advantage that much higher capacities may be attained especially when large quantities of coating material have to be applied. Typically a batch fluid bed is only capable of operating with a 100% increase in bed hold-up mass during the batch time i.e., the finished product has one kg of coating pr. kg initial material. A continuous fluid bed may be designed to accommodate any increase in coating mass.

It is therefore desirable to obtain a process which posses the advantages from both types of fluid bed coaters; is capable of handling large amounts of particles to be coated and which is capable of producing compositions of particles with a narrow particle size distribution even with a large increase in mass after coating.

Coating of particles is a time and space consuming process. Often it is necessary to recoat particles if the coated particles are not satisfactorily coated. In the search for a process which can provide means for coating large amounts of particles which needs to be coated more than once and furthermore provide control of a property related to the coating applied and defining whether or not the particles need more coating, we have developed the process of the invention.

Previously in order to obtain thick uniform coating of particles providing e.g., a mass increase of 500-1000% it would have been necessary to perform several batch coating processes getting at the most a total mass increase of 50 to 100% in each batch process step and resulting in a very narrow size distribution of the particles in each batch. Such a setup is, however, very time and space consuming thus increasing the cost of the final product.

One object of the present invention is to provide a process which provides a high throughput of coated particles. A second object of the present invention is to provide a process which is capable of producing coated particles with a specific property e.g., specific particle size.

The present invention comprises the attractive properties of both types of known fluid bed coaters such that it enables a process where large amounts of coating materials may be added.

### SUMMARY OF THE INVENTION

A first embodiment of the present invention is a process for coating of particles comprising the steps of:

a) feeding the particles to be coated to a coating unit wherein said particles are coated;

b) directly transferring the coated particles from the coating unit to a separator unit;

c) separating finished coated particles from unfinished coated particles;

d) removing the finished particles and transferring unfinished coated particles directly from the separator unit to a holding unit;

e) directly transferring the unfinished particles from the holding unit to the coating unit for re-coating.

The particles can repeat this cycle two times, three times, four times, five times or an infinite number of times if needed.

A second aspect of the present invention involves the finished particles that are obtained by the process of the invention.

A third aspect of the invention is a composition comprising the particles that is obtained by the process of the invention.

### BRIEF DESCRIPTION OF DRAWINGS

FIG. 1 shows a principal sketch of the invention comprising a coating unit, a separator unit and a holding unit.

FIG. 2 shows one embodiment of the invention comprising a coating unit in the form of a continuous fluid bed coater, a separator unit and a holding unit.

### DETAILED DESCRIPTION OF THE INVENTION

The present invention provides a useful method for controlling a specific property somehow related to the coating of a coated particle e.g., particle size, size distribution, color, taste, odor, activity, shape etc.

The following are examples of particles which can be obtained by the process of the present invention.

In the coating of proteins, e.g., enzymes it is very important that the coating thickness is well controlled so that a uniform coating thickness can be obtained. A uniform coating thickness will make possible the production of a granulate having a well defined specific activity and is therefore desirable. In the present context a “uniform coating” means that the variability of the relative coating mass between particles after coating is at the most 5% after a coating process resulting in an overall mass increase of more than 100%.

By the present process it is possible to coat large amounts of particles with a desired composition of coating materials which result in particles with a specific thickness and to reduce the time needed for doing so.

The process makes it possible to adjust the thickness of the coating and the particle size of the particles which gives granular compositions with very narrow particle size distributions, as it is possible to rerun the particles for recoating if the coating thickness is too low or the particle sizes are too small.

The process of the invention can be used with success when coating particles with a pigmented coating. It might be necessary to recoat some particles several times to obtain the same color throughout the batch. With the process of the present invention it is possible to separate the particles and transfer the particles which need more pigmented coating to a holding tank waiting for recoating.

When manufacturing particles comprising allergenic compounds, it is very important that the particles are coated sufficiently, resulting in low dust levels, this can be controlled by the process of the invention by checking the particles in the separator unit for activity and transferring the particles to the holding tank if the measured surface activity is too high. The process of the invention furthermore makes it possible to adjust the activity of the particles by controlling the added coating mass.

The process of the invention can furthermore be used for controlling taste and odour where there is a need for coating particles which has a bad taste or bad odour which needs to be masked. The coated particles are checked in the separator unit for the bad taste or odour and if the taste or odour is still present or significant the particles are transferred to the holding tank.

The Process

The process of the invention is a process for coating of particles, which consists of at least three units in connection. The coating unit which is a continuous coating unit is directly connected to a separator unit which is directly connected to a holding unit which act as a holding tank for particles which needs to be recoated. The word “directly” in the context of the present process means with a direct connection between the units, being e.g., a pipe, or a drying apparatus.

The process of the invention comprises the following steps:

a) feeding the particles to be coated to a coating unit wherein said particles are coated;

b) directly transferring the coated particles from the coating unit to a separator unit;

c) separating finished coated particles from unfinished coated particles;

d) removing the finished coated particles and transferring unfinished coated particles directly from the separator unit to a holding unit; and

e) directly transferring particles from the holding unit to the coating unit for re-coating.

Optionally a drying unit may be included in the process after the coating unit. In a particular embodiment of the present invention the process further comprises the following step:

Transferring coated particles from the coating unit to a drying unit, wherein the coated particles are dried and transferring the dried particles to the separator unit. The drying unit may be incorporated in the coating unit.

In a particular embodiment of the present invention the process of the invention comprises the following steps:

a) feeding the particles to be coated to a coating unit wherein said particles are coated;

b) drying the coated particles of a);

c) directly transferring the dried particles to a separator unit;

d) separating finished coated particles from unfinished coated particles;

e) removing the finished coated particles and transferring unfinished coated particles directly from the separator unit to a holding unit; and

f) directly transferring particles from the holding unit to the coating unit for re-coating.

The particles are transported between the process units by any suitable transporting device known in the art e.g., pneumatic transport, belt or other conveyer types.

The particles provided to the process can either be added to the coating unit or to the holding unit

In a particular embodiment the particles provided to the process is a batch of particles.

The process of the invention can be applied in order to provide almost any desired increase in total mass, wherein the total increase is set by the number of coating cycles applied.

In one embodiment the total mass increase of each particle is at least 100%, particularly at least 200%, more particularly at least 500%. In a particular embodiment of the present invention the total mass increase of the particles are at least 100%. In another particular embodiment the dispersion number in the coating unit is less than 0.5 and the total mass increase of the particles during the process is at least 100%.

The Coating Unit

The apparatus used as coating unit is an apparatus wherein the particles are transported through while being coated with a composition of coating materials. The apparatus can be any coating apparatus, e.g., a fluid bed, a top spray coater where the particles to be coated are suspended in/on a fluidized bed or a bottom spray coater where the coating liquid is sprayed up into a fluidized bed, a rotating drum or any other spray configuration known in the art. In a particular embodiment the coating unit is a continuous fluid bed coating unit, wherein the airflow of the fluid bed is adjusted to accommodate for the mass increase of the particles for each pass.

If the process is applied to a batch of particles the coating unit is defined by having a volume, the volume is the total volume of the coating unit that may contain particles, which is small compared to the total volume of the batch size, i.e., the sum of volumes in the coating unit and the holding unit. This implies that the volume in the coating unit is much smaller than the volume of the holding unit.

The particles are sprayed with a coating material in the coating unit where after the particles are removed and transferred to the separator unit.

In a particular embodiment the particles are coated in the coating unit at least two times. In another embodiment, the particles are coated in the coating unit at least three times. In a further embodiment, the particles are coated in the coating unit at least four times. In yet another embodiment, the particles are coated in the coating unit at least five times. In a further embodiment, the particles are coated in the coating unit an infinite number of times. In another particular embodiment the mass increase for the particles per passage of the coating unit is less than 10%.

In a particular embodiment the coating time in the coating unit for the particles in each pass is less than 5 min.

In one embodiment the mass increase for the particles per passage of the coating unit is less than 25%, particularly less than 15%, more particularly less than 5%.

In another embodiment the mass increase for each particle per passage of the coating unit is less than 25%, particularly less than 15%, more particularly less than 5%.

One possible execution of the coating unit without limiting the scope of invention is a rectangular fluid bed having a length several times its width, which will reduce the dispersion coefficient. Spray nozzles may be placed either above, below or within the fluidized layer.

Another possible execution of the coating unit without limiting the scope of invention is a drum- or pan coater, which will result in a high dispersion coefficient.

The dispersion number (Nd) in the coating unit is Nd=D/(uL), where D is the dispersion coefficient, u a characteristic velocity and L a characteristic length scale in the unit.

The process of the invention may comprise more than one coating unit.

The Separator Unit

The separator unit is an automatic separator unit, wherein the particles are checked for the property relating to whether or not the particles need to be re-coated, that is whether or not the particles are finished. The term “finished particles” as used in this application refers to particles that have attained the desired characteristic, such as the desired thickness of coating, the desired uniformity of the color of coating, etc. The term “unfinished particles” refers to particles that have not yet attained the desired characteristic, such as the desired thickness of coating or the desired uniformity of color of coating, etc.

The separator unit comprises some feature which enables the separator unit to identify and separate particles that are to be removed from the process (that is, particles that are finished) and particles that are to be transferred to the holding unit for re-coating because they are unfinished.

If separating by size—sieves, laser diffraction or vision systems may be used to identify which particles to remove and which particles to transfer to the holding unit. If checking for active dust—fluorescence spectroscopy may be used. If checking for taste and odour—head space gas chromatography methods may be used etc.

If the particles are found to be unsatisfactorily coated the particles are transferred to the holding unit, if the particles are found to be satisfactorily coated the particles are removed from the process e.g., for packing.

The separator unit may further comprise the feature of being able of identifying particles which somehow are outside a specified range and thereby being transferred for disposal or transferred for storage for subsequent processing.

In a particular embodiment the separator unit comprise the step of identifying and separating the particles in a product fraction, a recycle fraction and optionally a fraction of disposable particles.

In another particular embodiment of the present invention the particles may be classified into two or more fractions according to any useful property, such as size, colour, shape, active content or similar. One fraction is returned to the holding unit and the other fraction or fractions may be removed from the process, or returned to other holding units.

Using this feature of the invention thus enables the coating process to be significantly improved in both quality and capacity at the same time. This is due to the fact that it is needed to apply more than one coating to most particles because of the inherent differences in residence times in the spray zone of the particles. This is especially a problem in large scale coating equipment—even in ordinary batch coaters. The residence time in a batch coater is pr. definition the same, but a significant distribution in the real residence time in the spray zone exists.

The Holding Unit

The holding unit act as a variable size holding container, wherein the volume of the particles present may vary.

In a particular embodiment the volume of the coating unit, is less than the volume of the holding unit. The holding unit may be one big holding unit or consist of a number of smaller holding units. When referring to the volume of the holding unit it is meant the total volume in the one or more holding units used in the process.

In a further particular embodiment of the present invention the volume of the coating unit, V1, and the volume of the holding unit, V3, are selected so: V1<<V3, the volume of the coating unit is much less than the volume of the holding unit.

This enables the process to accommodate for a large variation in the total batch size if process is applied to a batch of particles without changing the residence time in the coating unit and thereby the coating efficiency.

In a particular embodiment of the present invention V3 is greater than two times V1. In a more particular embodiment V3 is greater than three times V1. In an even more particular embodiment of the present invention V3 is greater than five times V1. In a most particular embodiment of the present invention V3 is between two times V1 and ten times V1.

In a particular embodiment the residence time of the particles in the holding unit is at least 50 times the residence time of the particles in the coating unit.

The holding unit can be designed to have whatever dispersion number desired. If no or little dispersion is wanted the holding unit may be designed as a mass flow tank. If high dispersion is wanted the holding unit might comprise some sort of stirring aggregate. The process of the invention may comprise more than one holding unit.

Dispersion during the Process

The dispersion during the process is of some importance and may have a significant influence on the obtained coated particles. For some purposes it is important that the dispersion of particles is as low as possible during the process e.g., if a specific coating thickness is desired. For other purposes it may be a desire that the dispersion of particles is high e.g., to obtain a specific distribution of coating thicknesses within a batch of particles, which can be used to produce controlled release products.

The coating unit and the holding unit may be designed to fulfil whatever dispersion during the process of the particles wanted depending on what features of the coated product are desired.

Low Dispersion during the Process:

The Coating Unit:

If low dispersion of the particles is a desire, each particle in the coating unit should have the same residence time in the coating unit so the first added particles also are leaving the coating unit first and that any mixing of the particles within the unit is minimal, to ensure that the particles get coated with the same amount of coating material. The flow in the coating unit should resemble what is known in the art as a plug flow. This can also be expressed as the coating unit should have a low dispersion number, Nd<<1; (i.e., limited back-mixing).

As a consequence of a low dispersion number and a fast flow rate of particles through the coating unit, the amount of coating added to each particle at each pass of the coating unit will be small.

In one embodiment the dispersion number (Nd) in the coating unit according to formula Nd=D/(uL) is less than 0.05, particularly less than 0.005, more particularly less than 0.001. In a particular embodiment the dispersion number in the coating unit is less than 0.05.

In one embodiment of the present invention the activity strength (i.e., content of an active ingredient) of a granular composition is adjusted. The present invention adjust the activity by applying a continuous coating unit wherein the residence time for each particle comprising an active compound is very short and having a fast flow rate of particles through the coating unit thereby avoiding back mixing of particles, so that the particles, which enter the unit first will be the first to leave the unit. The residence time distribution thus provides a uniform coating of each particle and any desired coating thickness can be obtained by adding small amounts of coating for each passing of the coating unit and recycling each particle between the separator unit, the holding unit and the coating unit for as many cycles as necessary.

The Holding Unit:

If the dispersion in the holding unit is very low the particles entering the holding unit first are leaving the holding unit first.

The holding unit is characterised by a vessel dispersion number Nd=D/(uL), where D is the dispersion coefficient, u a characteristic velocity and L a characteristic length scale in the unit; is small:
Nd<<1; (i.e., limited back-mixing);

In one embodiment the dispersion number (Nd) in the holding unit according to formula Nd=D/(uL) is less than 0.05, particularly less than 0.005, more particularly less than 0.001. In a particular embodiment the dispersion number in the holding unit is less than 0.05.

In a particular embodiment of the present invention the invention relates to the use of a continuous coating unit, a separator unit and a storage unit connected in loop for the uniform coating of a batch of particles, wherein the residence time of the particles in the storage unit is at least fifty times the residence time of the particles in the coating unit, the dispersion number (Nd) in the coating unit is less than 0.5 and the total mass increase of the particles during the process is at least 100%.

High Dispersion during the Process:

The Coating Unit

In a particular embodiment, the particles have different residence time in the coating unit. A possibility is to recycle particles in the coating unit to obtain a well defined residence time distribution which in turn will lead to a well defined distribution of coating thicknesses. This may be used to manufacture controlled release products because different coating thicknesses will lead to different release rates.

In a particular embodiment the dispersion number in the coating unit is more than 0.2.

The Holding Unit

If high dispersion is wanted a possibility is to use a stirring aggregate within the holding tank or design the tank so that some degree of funnel flow exists.

In a particular embodiment of the present invention the dispersion number in the holding unit is more than 0.2.

The Particles of the Invention

The present invention further relates to finished coated particles obtainable by the process of the invention.

The finished particles of the process comprise a core particle and a coating. The core particle and/or the coating may comprise an active compound.

The particle sizes of the finished particles may be from about 50 micrometers to about 2000 micrometers; more particular from about 100 micrometers to about 1000 micrometers; even more particular from about 200 micrometers to about 700 micrometers; and most particularly from about 250 micrometers to about 500 micrometers.

The Coating

The coatings may be from 1 micron to 1500 microns thick. In a particular embodiment the coating is more than 30 microns thick, in a more particular embodiment the coating is more than 50 microns thick, in a most particular embodiment the coating is more than 75 microns thick. In a particular embodiment the coating is less than 1000 microns thick, in a more particular embodiment the coating is less than 500 microns thick, in an even more particular embodiment the coating is less than 250 microns thick, in a most particular embodiment the coating is less than 150 microns thick.

The coating material may or may not comprise an active compound. In a particular embodiment of the present invention the coating does not comprise an active compound.

Besides the coating equipment the coating material may have a large impact on the coating capacity and the coating quality. The coating material is a liquid formulation and can be a solution, a suspension, an emulsion or a melt.

Conventional coating materials as known to the art may suitably be used, such as materials described in WO 89/08694, WO 89/08695, EP 270 608 B1 and/or WO 00/01793. Other examples of conventional coating materials may be found in U.S. Pat. No. 4,106,991, EP 170360, EP 304332, EP 304331, EP 458849, EP 458845, WO 97/39116, WO 92/12645A, WO 89/08695, WO 89/08694, WO 87/07292, WO 91/06638, WO 92/13030, WO 93/07260, WO 93/07263, WO 96/38527, WO 96/16151, WO 97/23606, U.S. Pat. No. 5,324,649, U.S. Pat. No. 4,689,297, EP 206417, EP 193829, DE 4344215, DE 4322229 A, DD 263790, JP 61162185 A and/or JP 58179492.

The coating may comprise but are not limited to materials selected from binders, waxes, synthetic polymers, polysaccharides, solvents, fibers, fillers, salts, water insoluble minerals, pigments, dyes, enzyme stabilizers or combinations thereof.

Particles to be Coated

The particles to be coated in the context of the present invention are the particles feed to the process. The particles to be coated may be non-active particles or particles comprising active compounds. In a particular embodiment of the present invention the particle cores to be coated comprise an active compound. The particles are all prepared in advance before they are added to the coating process.

The particles to be coated are at least 20 microns; more particularly the particles to be coated are at least 40 microns; even more particularly the particles to be coated are 60 microns; most particularly the particles to be coated are 100 microns. In a particular embodiment the particles to be coated are 50 microns to 400 microns.

In a particular embodiment of the present invention the particles to be coated are less than 600 microns. In a more particular embodiment the particles to be coated are less than 500 microns. In an even more particular embodiment of the present invention the particles to be coated are less than 400 microns. In a most particular embodiment of the present invention the particles to be coated are less than 300 microns. Any conventional methods and materials may be used to prepare the particles to be coated.

The particles may be coated from 1 to 50 times; more particular from 2 to 25 times; even more particular from 2 to 10 times.

Examples of known conventional cores and materials to be used are, inter alia, described in, U.S. Pat. No. 4,106,991 (in particular), EP 170360, EP 304332, EP 304331, EP 458849, EP 458845, WO 01/25412, WO 97/39116, WO 92/12645, WO 89/08695, WO 89/08694, WO 87/07292, WO 91/06638, WO 92/13030, WO 93/07260, WO 93/07263, WO 96/38527, WO 96/16151, WO 97/23606, U.S. Pat. No. 5,324,649, U.S. Pat. No. 4,689,297, EP 206417, EP 193829, DE 4344215, DE 4322229 A, JP 61162185 A, JP 58179492, WO 04/33083, PCT/DK01/00627.

Non-Active Particles to be Coated

Non active particles such as carrier particles, seed particles, placebo particles or non-pareil particles are particles not comprising active compounds upon which a coating mixture comprising the active compound can be layered. They may be formulated with organic or inorganic materials, such as inorganic salts, sugars, sugar alcohols, small organic molecules such as organic acids or salts, starch, cellulose, polysaccharides, minerals such as clays or silicates or a combination of two or more of these.

In a particular embodiment of the present invention the particles to be coated with an active comprising coating material are non-active particles.

Active Compounds

The active compound of the present invention either present in the core or in the coating may be any active compound or mixture of active compounds, which benefits from being separated from the environment surrounding the granule. The term “active” is meant to encompass all compounds, which upon release from the coated particle upon applying the coated particle of the invention in a process, serve a purpose of improving the process. The active compound may be inorganic of nature or organic of nature. Particularly active compounds are active biological compounds which are usually very sensitive to the surrounding environment such as compounds obtainable from microorganisms. More particularly active compounds are peptides or polypeptides or proteins. Most particularly active compounds are proteins such as enzymes. Further suitable active compounds are bleaches, growth promoters, antibiotics, antigenic determinants to be used as vaccines, polypeptides engineered to have an increased content of essential amino acids, hormones and other therapeutic proteins. In a particular embodiment of the present invention the particles to be coated comprise proteins. In a more particular embodiment the proteins are enzymes.

The enzyme in the context of the present invention may be any enzyme or combination of different enzymes. Accordingly, when reference is made to an “enzyme” this will in general be understood to include one enzyme or a combination of enzymes.

It is to be understood that enzyme variants (produced, for example, by recombinant techniques) are included within the meaning of the term “enzyme”. Examples of such enzyme variants are disclosed in, e.g., EP 251,446 (Genencor), WO. 91/00345 (Novo Nordisk), EP 525,610 (Solvay) and WO 94/02618 (Gist-Brocades NV).

Enzymes can be classified on the basis of the handbook Enzyme Nomenclature from NC-IUBMB, 1992), see also the ENZYME site at the internet: www.expasv.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes, such as endoglucanase, xylanase, galactanase, mannanase, dextranase and alpha-galactosidase, in families based on amino acid sequence similarities has been proposed a few years ago. They currently fall into 90 different families: See the CAZy(ModO) internet site (Coutinho, P. M. & Henrissat, B. (1999) Carbohydrate-Active Enzymes server at URL: afmb.cnrs-mrs.fr/˜cazy/CAZY/index.html (corresponding papers: Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12; Coutinho, P. M. & Henrissat, B. (1999) The modular structure of cellulases and other carbohydrate-active enzymes: an integrated database approach. In “Genetics, Biochemistry and Ecology of Cellulose Degradation”., K. Ohmiya, K. Hayashi, K. Sakka, Y. Kobayashi, S. Karita and T. Kimura eds., Uni Publishers Co., Tokyo, pp. 15-23).

The types of enzymes which may be incorporated in particles of the invention include oxidoreductases (EC 1.-.-.-), transferases (EC 2.-.-.-), hydrolases (EC 3.-.-.-), lyases (EC 4.-.-.-), isomerases (EC 5.-.-.-) and ligases (EC 6.-.-.-).

Preferred oxidoreductases in the context of the invention are peroxidases (EC 1.11.1), laccases (EC 1.10.3.2) and glucose oxidases (EC 1.1.3.4)]. An example of a commercially available oxidoreductase (EC 1.-.-.-) is Gluzyme□(enzyme available from Novozymes A/S). Further oxidoreductases are available from other suppliers. Preferred transferases are transferases in any of the following sub-classes:

a. Transferases transferring one-carbon groups (EC 2.1);

b. transferases transferring aldehyde or ketone residues (EC 2.2); acyltransferases (EC 2.3);

c. glycosyltransferases (EC 2.4);

d. transferases transferring alkyl or aryl groups, other that methyl groups (EC 2.5); and

e. transferases transferring nitrogeneous groups (EC 2.6).

A most preferred type of transferase in the context of the invention is a transglutaminase (protein-glutamine gamma-glutamyltransferase; EC 2.3.2.13).

Further examples of suitable transglutaminases are described in WO 96/06931 .(Novo Nordisk A/S).

Preferred hydrolases in the context of the invention are: carboxylic ester hydrolases (EC 3.1.1.-) such as lipases (EC 3.1.1.3); phytases (EC 3.1.3.-),- e.g., 3-phytases (EC 3.1.3.8) and 6-phytases (EC 3.1.3.26); glycosidases (EC 3.2, which fall within a group denoted herein as “carbohydrases”), such as alpha-amylases (EC 3.2.1.1); peptidases (EC 3.4, also known as proteases); and other carbonyl hydrolases. Examples of commercially available phytases include Bio-Feed™Phytase (Novozymes), Ronozyme™P (DSM Nutritional Products), Natuphos™(BASF), Finase™(AB Enzymes), and the Phyzyme™product series (Danisco). Other preferred phytases include those described in WO 98/28408, WO 00/43503, and WO 03/066847.

In the present context, the term “carbohydrase” is used to denote not only enzymes capable of breaking down carbohydrate chains (e.g., starches or cellulose) of especially five-and six-membered ring structures (i.e., glycosidases, EC 3.2), but also enzymes capable of isomerizing carbohydrates, e.g., six-membered ring structures such as D-glucose to five-membered ring structures such as D-fructose.

Carbohydrases of relevance include the following (EC numbers in parentheses): alpha-amylases (EC 3.2.1.1), beta-amylases (EC 3.2.1.2), glucan 1,4-alpha-glucosidases (EC 3.2.1.3), endo-1,4-beta-glucanase (cellulases, EC 3.2.1.4), endo-1,3(4)-beta-glucanases (EC 3.2.1.6), endo-1,4-beta-xylanases (EC 3.2.1.8), dextranases (EC 3.2.1.11), chitinases (EC 3.2.1.14), polygalacturonases (EC 3.2.1.15), lysozymes (EC 3.2.1.17), beta-glucosidases (EC 3.2.1.21), alpha-galactosidases (EC 3.2.1.22), beta-galactosidases (EC 3.2.1.23), amylo-1,6-glucosidases (EC 3.2.1.33), xylan 1,4-beta-xylosidases (EC 3.2.1.37), glucan endo-1,3-beta-D-glucosidases (EC 3.2.1.39), alpha-dextrin endo-1,6-alpha-glucosidases (EC3.2.1.41), sucrose alpha-glucosidases (EC 3.2.1.48), glucan endo-1,3-alpha-glucosidases (EC 3.2.1.59), glucan 1,4-beta-glucosidases (EC 3.2.1.74), glucan endo-1,6-beta-glucosidases (EC 3.2.1.75), galactanases (EC 3.2.1.89), arabinan endo-1,5-alpha-L-arabinosidases (EC 3.2.1.99), lactases (EC 3.2.1.108), chitosanases (EC 3.2.1.132) and xylose isomerases (EC 5.3.1.5).

Examples of commercially available proteases (peptidases) include Kannase™, Everlase™, Esperase□, Alcalase□, Neutrase□, Durazymo□, Savinase®, Ovozyme®, Pyrase®, Pancreatic Trypsin NOVO (PTN), Bio-Feed□ Pro and Clear-Lens □ Pro (all available from Novozymes A/S, Bagsvaerd, Denmark). Other preferred proteases include those described in WO 01/58275 and WO 01/58276.

Other commercially available proteases include Ronozyme^{□□} Pro, Maxatase®, Maxacal®, Maxapem®, Opticlean®, Properase®, Purafect® and Purafect Ox® (available from Genencor International Inc., Gist-Brocades, BASF, or DSM Nutritional Products).

Examples of commercially available lipases include Lipex®, Lipoprime™, Lipopan® Lipolase®, Lipolase® Ultra, Lipozyme®, Palatase®, Resinase®, Novozym® 435 and Lecitase® (all available from Novozymes A/S).

Other commercially available lipases include Lumafast® (Pseudomonas mendocina lipase from Genencor International Inc.); Lipomax® (Ps. pseudoalcaligenes lipase from Gist-Brocades/Genencor Int. Inc.; and Bacillus sp. lipase from Solvay enzymes. Further lipases are available from other suppliers.

Examples of commercially available carbohydrases include Alpha-Gal®, Bio-Feed® Alpha, Bio-Feed® Beta, Bio-Feed® Plus, Bio-Feed® Wheat, Bio-Feed® Z Novozyme® 188, Carezyme® Celluclaste®, Cellusoft®, Celluzymeo®, Ceremyl®, Citrozym®, Denimax®, Dezyme®, Dextrozyme®, Duramyl®, Energex®, Finizym®, Fungamyl®, Gamanase®, Glucanex®, Lactozym®, Liquezyme®, Maltogenase®, Natalase®, Pentopan®, Pectinex®, Promozyme®, Pulpzyme®, Novamyl®, Termamyl®, AMGO® (Amyloglucosidase Novo), Maltogenase®, Sweetzyme® and Aquazym® (all available from Novozymes A/S). Further carbohydrases are available from other suppliers, such as the Roxazyme® and Ronozyme® product series (DSM Nutritional Products), the Avizyme®, Porzyme® and Grindazyme® product series (Danisco, Finnfeeds), and Natugrain® (BASF), Purastar® and Purastar® OxAm (Genencor).

Other commercially available enzymes include Mannaway®, Pectaway®, Stainzyme® and Renozyme®.

Lipases: Suitable lipases include those of bacterial or-fungal origin. Chemically modified or protein engineered mutants are included. Examples of useful lipases include lipases from Humicola (synonym Thermomyces), e.g., from H. lanuginosa (T Ianuginosus) as described in EP 258 068 and EP 305 216 or from H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens , Pseudomonas sp. strain SD 705 (WO 95/06720 and WO 96/27002), P. wisconsinensis (WO 96/12012), a Bacillus lipase, e.g., from B. subtilis (Dartois et al. (1993), Biochemica et Biophysica Acta, 1131, 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Examples of commercially available lipases include LIPEX, LIPOPRIMETM™, LIPOLASETM™, LIPOLASETM™ Ultra, LIPOZYMETM™, PALATASETM™, NOVOZYMTM™ 435 and LECITASETM™ (all available from Novozymes A/S).

Other commercially available lipases include LUMAFAS™ (Pseudomonas mendocina lipase from Genencor International Inc.); LIPOMAXTM™ (Ps. pseudoalcaligenes lipase from DSM/Genencor Int. Inc.; and Bacillus sp. lipase from Genencor enzymes. Further lipases are available from other suppliers.

Examples of commercially available carbohydrases include ALPHA-GAL™, BIO-FEED™ Alpha, BIO-FEEDTM™ Beta, BIO-FEED™ Plus, BIO-FEED™ Plus, NOVOZYMETM™ 188, CELLUCLAS™, CELLUSOFT™, CEREMYL™, CITROZYM™, DENIMAX™, DEZYME™, DEXTROZYME™, FINIZYM™, FUNGAMYL™, GAMANASE™, GLUCANEX™, LACTOZYMTM™, MALTOGENANO: 9SE™, PENTOPAN™, PECTINEX™, PROMOZYME™, PULPZYMETM™, NOVAMYL™, TERMAMYL™, AMG™ (Amyloglucosidase Novo), MALTOGENASE™, SWEETZYME™and AQUAZYM™ (all available from Ndvozymes A/S). Further carbohydrases are available from other suppliers.

Amylases: Suitable amylases (alpha and/or beta) include those of bacterial or fungal origin. Chemically modified or protein engineered mutants are included. Amylases include, for example, alpha-amylases obtained from Bacillus, e.g., a special strain of B. lichenifonmis, described in more detail in GB 1,296,839.

Examples of useful amylases are the variants described in WO 94/02597, WO 94/18314, WO 96/23873, and WO 97/43424, especially the variants-with.substitutions in one or more of the following positions: 15, 23,105,106, 124, 128, 133, 154, 156,181, 188, 190, 197, 202, 208, 209, 243, 264, 304, 305, 391, 408, and 444.

Commercially available amylases are NATALASE™ STAINZYME™, DURAMYL□, TERMAMYL®, TERMAMYL™ ULTRA, FUNGAMYL® and BAN® (Novozymes A/S), RAPIDASE®, PURASTAR® and PURASTAR OXAM® (from Genencor International Inc.).

Cellulases: Suitable cellulases include those of.bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thennophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, and WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Commercially available cellulases include CELLUZYME®, ENDOLASE®, RENOZYME® and CAREZYME® (Novozymes A/S), CLAZINASE®, and PURADAX HA® (Genencor International Inc.), and KAC-500(B) ® (Kao Corporation).

Oxidoreductases: Particular oxidoreductases in the context of the invention are peroxidases (EC 1.11.1), laccases (EC 1.10.3.2) and glucose oxidases (EC 1.1.3.4)]. An Example of a commercially available oxidoreductase (EC 1.-.-.-) is GLUZYME™ (enzyme available from Novozymes A/S). Further oxidoreductases are available from other suppliers.

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include GUARDZYME® (Novozymes A/S).

Mannanase: Any mannanase suitable for use in alkaline solutions can be used. Suitable mannanases include those of bacterial or fungal origin. Chemically or genetically modified mutants are included.

In a preferred embodiment the mannanase is derived from a strain of the genus Bacillus, especially Bacillus sp. 1633 disclosed in positions 31-330 of $EQ ID NO:2 or in SEQ ID NO: 5 of WO 99/64619 or Bacillus agaradhaerens, for example from the type strain DSM 8721. In a more preferred embodiment of the present invention the mannanase is derived from Alkalophilic Bacillus.

Suitable mannanases include MANNAWAYO® (Novozymes A/S).

Pectate lyase: Any pectate lyase suitable for use in alkaline solutions can be used. Suitable pectate lyases include those of bacterial or fungal origin. Chemically or genetically modified mutants are included.

In a preferred embodiment the pectate lyase is derived from a strain of the genus Bacillus, especially a strain of Bacillus substilis, especially Bacillus subtilis DSM14218 disclosed in SEQ ID NO: 2 or a variant thereof disclosed in Example 6 of WO 02/092741. In a more preferred embodiment of the present invention the pectate lyase is derived from Bacillus licheniformis.

Use of the Coated Particles of the Invention

The invention further relates to the use of the particles of the process.

The particles of the invention are suitable for use in compositions comprising coated particles.

The particles of the invention may be used within the pharmaceutical area, in detergent compositions for cleaning an object, in textile production, in baking for improving bread, in feed compositions for improving the feed and in food products, in personal care products etc.

Accordingly the compositions may be a detergent composition, a pharmaceutical composition, a feed composition, a food composition, a baking composition or an additive to be incorporated in such compositions.

In a particular embodiment the composition is a detergent. In another particular embodiment the composition is a feed. In a further embodiment the composition is a food.

The invention further relates to a method comprising providing the particles of the invention to a liquid for cleaning an object or to an animal for improving its digestion or to a dough for improving a bread.

### EXAMPLES

### Example 1

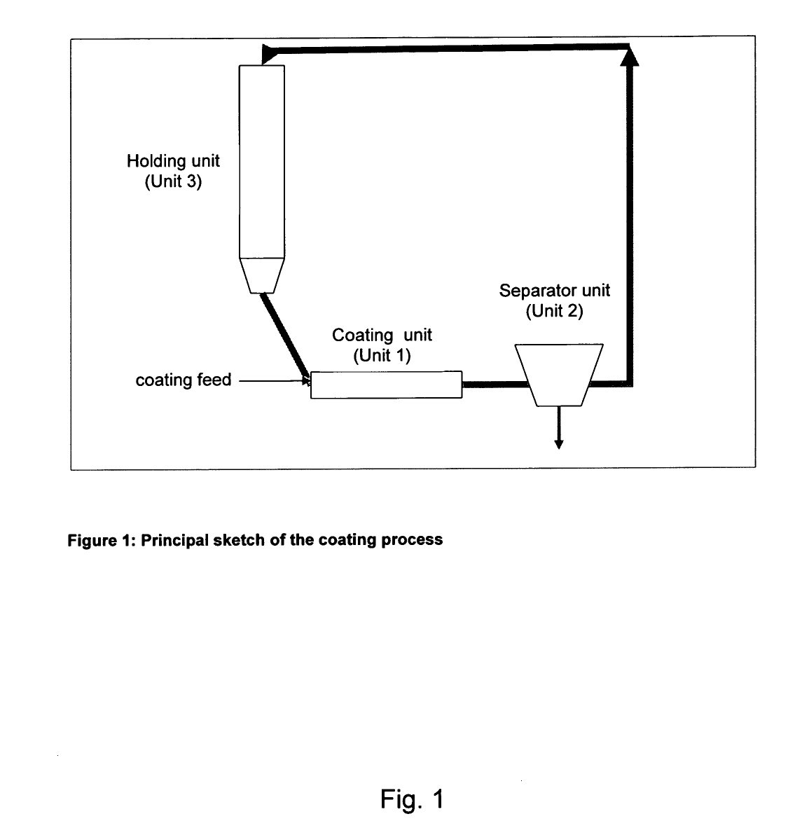

A sketch of the coating process comprising a coating unit, a separator unit and a holding unit is shown in FIG. 1.

### Example 2

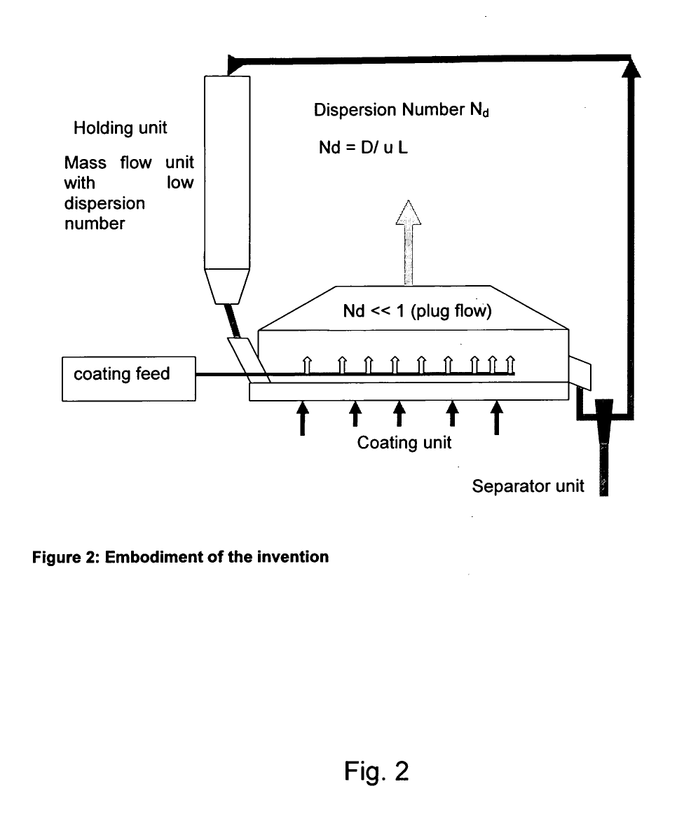

A non limiting sketch of an embodiment of the invention is shown in FIG. 2. A typical industrial coating unit according to the present invention might have the following design. The coating unit shown below is a rectangular fluid bed having a width of 0.5 m and a length of 4 m. The coating layer will be small, i.e., 0.2 to 0.4 m. This design will insure a low dispersion coefficient. If the fluid bed is operated so the re-circulation speed is high so the average linear velocity of the product transported through the unit is at least u=2 m/min we will obtain a very low dispersion number Nd for the unit. The recirculation rate is equal to the product of the velocity u and the fluid bed width and the product filling height in the coating unit.

The volume in the coating unit will thus be about 0.4 to 0.8 m³. The volume of the holding unit will typically be 4-8 m³, which will allow for coating volumes up to about 1000%. In the present example the recirculation speed will be 100-200 liters/min., which corresponds to a residence time in the coating unit of 2-4 min. Typical operating parameters are shown in the following table:

TABLE 1

| Parameter | Value | Unit |
|---|---|---|
| Inlet temperature | 130 | ° C. |
| Air volume | 10000 | Nm3/h |
| Spray rate of a 60% DM coating feed | 800 | Kg/h |
| No. nozzles | 12 | |

Various references are cited herein, the disclosures of which are incorporated by reference in their entireties.

1. A process for coating particles comprising the steps of:
a) feeding particles to a coating unit wherein said particles are coated;
b) directly transferring coated particles from the coating unit to a separator unit;
c) separating finished coated particles from unfinished coated particles;
d) removing the finished coated particles and transferring the unfinished coated particles directly from the separator unit to a holding unit, and
e) directly transferring unfinished coated particles from the holding unit to the coating unit for re-coating.

2. The process according to claim 1, wherein the particles provided to the process is a batch of particles.

3. The process according to claim 1, wherein the volume of the coating unit, is less than the volume of the holding unit.

4. The process according to claim 2, wherein the volume of the holding unit is greater than two times the volume of the coating unit.

5. The process according to claim 1, wherein the particles are coated in the coating unit at least two times.

6. The process according to claim 1, wherein the mass increase for the particles per passage of the coating unit is less than 10%,

7. The process according to claim 1, wherein the coating time in the coating unit for the particles in each pass is less than 5 min.

8. The process according to claim 1, wherein the dispersion number in the coating unit is less than 0.05.

9. The process according to claim 1, wherein the dispersion number in the coating unit is ore than 0.2.

10. The process according to claim 1, wherein the dispersion number in the holding unit is less than 0.05.

11. The process according to claim 1, wherein the dispersion number in the holding unit is more than 0.2.

12. The process according to claim 1, wherein the coating unit is a continuous fluid bed coating unit.

13. The process according claim 12, wherein the airflow of the fluid bed is adjusted to accommodate for the mass increase of the particles for each pass.

14. The process according to claim 1, wherein the total mass increase of the particles are at least 100%.

15. The process of claim 1, wherein the residence time of the particles in the holding unit is at least 50 times the residence time of the particles in the coating unit.

16. The process of claim 1, wherein the dispersion number in the coating unit is less than 0.5 and the total mass increase of the particles during the process is at least 100%.

17. The process according to claim 1, wherein the particles to be coated in the process is added to either the coating unit or the holding unit.

18-20. (canceled)

21. A finished coated particle obtainable by a process according to claim 1.

22. A composition comprising a coated particle of claim 21.

23-28. (canceled)

29. A process coating particles comprising the steps of:
a) feeding the particles to be coated to a coating unit wherein said particles are coated;
b) drying the coated particles of a);
c) directly transferring the dried particles to a separator unit;
d) separating finished coated particles from unfinished coated particles;
e) removing the finished coated particles and transferring unfinished coated particles directly from the separator unit to a holding unit;
f) directly transferring particles from the holding unit to the coating unit for re-coating.
